(19) United States
(12) Patent Application Publication
(10) Pub. No.: US 2025/0154580 A1
(43) Pub. Date: May 15, 2025

(54) ENZYME TRANSLOCATORS IN NANOGAP WITH 3' -ESTERS

(71) Applicant: Robert Bosch GmbH, Stuttgart (DE)

(72) Inventors: Nadezda FOMINA, Redwood City, CA (US); Christopher JOHNSON, San Carlos, CA (US); Young Shik SHIN, Mountain View, CA (US); Christoph LANG, Sunnyvale, CA (US); Armin DARVISH, Daly City, CA (US); Gary YAMA, Mountain View, CA (US); Abhi BHADRAN, Dallas, TX (US); Christian GRUMAZ, Waiblingen (DE); Sebastian PILSL, Rutesheim (DE); Gabrielle VUKASIN, San Jose, CA (US)

(73) Assignee: Robert Bosch GmbH, Stuttgart (DE)

(21) Appl. No.: 18/507,587

(22) Filed: Nov. 13, 2023

(51) Int. Cl. C12Q 1/6869; C12N 9/12; C12Q 1/00; G01N 27/06

(52) U.S. Cl. CPC C12Q 1/6869; C12N 9/1241; C12Q 1/001; G01N 27/06

(57) ABSTRACT

A method for nucleic acid sequencing includes providing at least one device comprising a first electrode and a second electrode separated by a dielectric layer, and a polymerase enzyme attached to the surface of the dielectric layer. The dielectric layer induces an electroactive molecule to interact with the electrodes to complete an electrical circuit. The polymerase enzyme targets a polynucleotide strand to the dielectric layer. A sample including a polynucleotide strand and modified nucleotides having an electroactive label covalently bound to the 3′—OH of a sugar ring of the nucleotide via an ester group is provided to the at least one device. Potentials are applied to each electrode to induce electron flow between the electrodes to produce a measurable electrical signal when an electroactive label is present in the dielectric layer. Electrical signals from the electrodes are detected to determine when a modified nucleotide is present in the dielectric layer.

### TECHNICAL FIELD

In at least one aspect, the present disclosure relates to systems, devices, and methods for nucleic acid sequencing.

### BACKGROUND

Single base resolution DNA sequencing is a significant goal within biotechnology. To date, most techniques require either significant rebuilding of the sequence from small reads or repeated runs to achieve fidelity. There is a need for technologies that combine the scalability and speed of semiconductor-based electrical detection with the high accuracy of SbS technologies to provide long reads at single base pair resolution.

### SUMMARY

In various aspects, a method for nucleic acid sequencing is provided. The method includes providing at least one device comprising a first electrode and a second electrode separated by a dielectric layer. The dielectric layer defines a sensing zone between the first electrode and the second electrode. A polymerase enzyme is attached to the surface of the dielectric layer. The polymerase enzyme targets a polynucleotide strand to the dielectric layer. The method further includes providing to the at least one device a sample including a polynucleotide strand and at least one modified nucleotide, the modified nucleotide having an electroactive label covalently bound to 3′-OH of a sugar ring of the nucleotide via an ester group; applying a first potential to the first electrode and a second potential to the second electrode to induce electron flow between the first and second electrodes to produce a measurable electrical signal when an electroactive label is present in the sensing zone; and detecting the electrical signals from the first and second electrodes to determine when a modified nucleotide is present in the sensing zone.

In additional aspects, a system for nucleic acid sequencing is provided. The system comprises at least one device including: at least one electrochemical nanoelectrode sensor comprising a first electrode, a second electrode, and a dielectric layer defining a sensing zone between the first electrode and the second electrode. A polymerase enzyme is attached to the surface of the dielectric layer, and the polymerase enzyme targets a polynucleotide strand to the sensing zone. The system additionally includes a controller configured to: direct a first current through the first electrode and a second current through the second electrode to induce electron flow between the first and second electrodes to produce a measurable electrical signal when an electroactive label is present in the sensing zone; provide to the at least one device a sample including a polynucleotide strand and at least one modified nucleotide, the modified nucleotide having an electroactive label covalently bound to 3′-OH of a sugar ring of the nucleotide via an ester group; and detect the first current flowing from the first electrode and the second current flowing from the second electrode.

In yet other aspects, a method for forming a system for nucleic acid sequencing is provided. The method comprises the steps of: providing a first electrode and a second electrode; positioning a dielectric layer between the first and second electrodes; attaching a polymerase enzyme to the surface of the dielectric layer, wherein the polymerase enzyme incorporates a modified nucleotide having an electroactive label covalently bound to 3′-OH of a sugar ring of the nucleotide via an ester group into a polynucleotide strand; and configuring the electrodes detect changes in current when an electroactive label is present within the sensing zone.

### BRIEF DESCRIPTION OF THE DRAWINGS

FIG. 1 illustrates the concept of sequencing using an electrochemical sensor, an immobilized polymerase, and labeled nucleotides.

FIG. 2 illustrates a schematic of 3 different geometries for modification of the planar electrode pair.

FIG. 3 illustrates a schematic of 2 different geometries of a non-planar design, where the electrode pairs are fabricated as a stack in a well format (A), and where the well is filled on one side (B).

FIG. 4 illustrates a method for synthesizing 3′-esterified dNTPs with electroactive labels (E).

FIG. 5 illustrates examples of electroactive labels (E) and linkers (L).

FIG. 6 illustrates a first step of a method for synthesizing a ferrocene-labeled dUTP reversible terminator with an ester group (A3).

FIG. 7 illustrates a second step of the method in FIG. 6 (synthesizing compound A2 from A1).

FIG. 8 illustrates a third step of the method in FIG. 6 (synthesizing compound A3 from A2).

FIG. 9 illustrates a first step of a method for synthesizing a ferrocene-labeled dUTP reversible terminator with an ester group (B5).

FIG. 10 illustrates a second step of the method in FIG. 9 (synthesizing compound B2 from B1).

FIG. 11 illustrates a third step of the method in FIG. 9 (synthesizing compound B3 from B2).

FIG. 12 illustrates a fourth step of the method in FIG. 9 (synthesizing compound B4 from B3).

FIG. 13 illustrates a fifth step of the method in FIG. 9 (synthesizing compound B5 from B4).

FIG. 14 illustrates a method for synthesizing a ferrocene-labeled dTTP reversible terminator with an ester group and no linker (C3).

FIG. 15 illustrates cyclic voltammograms (CV) of electroactive labels: anthraquinone carboxylic acid (green), methylene blue (blue), ferrocene carboxylic acid (orange), and phenothiazine carboxylic acid (red). The CVs are recorded in 10 mM Phosphate buffer solution containing 100 mM KCl and 10% DMSO.

FIG. 16 illustrates an embodiment of readout electronics shown for one electrode.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “polynucleotide”, “nucleotide”, “nucleotide sequence”, “nucleic acid” and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

The terms “complementarity” or “complement” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

The term “translocator”, “translocating protein”, “enzyme”, and “protein” as used herein refers to any peptide, oligopeptide, polypeptide, gene product, expression product, or protein capable of translocating a polynucleotide strand. Examples of proteins capable of translocating a polynucleotide strand include DNA polymerase, RNA polymerase, ribosome, a single-stranded binding protein, topoisomerase, helicase, nuclease, exonuclease, endonuclease, a zinc finger nuclease, an RNA guided DNA endonuclease, a transcription activator-like effector nuclease, a CRISPR protein, and combinations thereof.

Unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″)₂, —N(R′R″R′″)₃⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; single letters (e.g., “n” or “o”) are 1, 2, 3, 4, or 5; in the compounds disclosed herein a CH bond can be substituted with alkyl, lower alkyl, C₁₋₆ alkyl, C₆₋₁₀ aryl, C₆₋₁₀ heteroaryl, —NO₂, —NH₂, —N(R′R″)₂, —N(R′R″R′″)₃⁺L″, Cl, F, Br, —CF₃, —CCl₃, —CN, —SO₃H, —PO₃H₂, —COOH, —CO₂R′, —COR′, —CHO, —OH, —OR′, —O-M⁺, —SO₃⁻M⁺, —PO₃⁻M⁺, —COO-M⁺, —CF₂H, —CF₂R′, —CFH₂, and —CFR′R″ where R′, R″ and R′″ are C₁₋₁₀ alkyl or C₆₋₁₈ aryl groups; the indication of a moiety or structure with positive charges implies that one or more negative counter ions are present to balance the charge, similarly, the indication of a moiety or structure with negative charges implies that one or more positive counter ions are present to balance the charge; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” as used herein means C₁₋₂₀, linear, branched, rings, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and alkenyl groups. “Lower alkyl” refers to an alkyl group having 1 to about 8 carbon atoms (i.e., a C₁₋₈ alkyl), e.g., 1, 2, 3, 4, 5, 6, 7, or 8 carbon atoms. Lower alkyl can also refer to a range between any two numbers of carbon atoms listed above. “Higher alkyl” refers to an alkyl group having about 10 to about 20 carbon atoms, e.g., 10, 11, 12, 13, 14, 15, 16, 17, 18, 19, or 20 carbon atoms. Higher alkyl can also refer to a range between any two number of carbon atoms listed above.

The term “aryl” as used herein means an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether. Examples of aryl include, but are not limited to, phenyl, naphthyl, biphenyl, and diphenylether, and the like. Aryl groups include heteroaryl groups, wherein the aromatic ring or rings include a heteroatom (e.g., N, O, S, or Se). Exemplary heteroaryl groups include, but are not limited to, furanyl, pyridyl, pyrimidinyl, imidazoyl, benzimidazolyl, benzofuranyl, benzothiophenyl, quinolinyl, isoquinolinyl, thiophenyl, and the like. The aryl group can be optionally substituted (a “substituted aryl”) with one or more aryl group substituents, which can be the same or different, wherein “aryl group substituent” includes alkyl (saturated or unsaturated), substituted alkyl (e.g., haloalkyl and perhaloalkyl, such as but not limited to —CF₃), cycloalkyl, aryl, substituted aryl, aralkyl, halo, nitro, hydroxyl, acyl, carboxyl, alkoxyl (e.g., methoxy), aryloxyl, aralkyloxyl, thioalkyl, thioaryl, thioaralkyl, amino (e.g., aminoalkyl, aminodialkyl, aminoaryl, etc.), sulfonyl, and sulfinyl.

The terms “redox molecule”, “redox label”, “electroactive molecule”, and “electroactive label” may all be used interchangeably to refer to a molecule that is a redox species capable of undergoing reversible oxidation-reduction reactions under applied electrical potential.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1, to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

In this application, electroactive molecules include Redox molecules, a Redox signal includes electrical signals such as a change in current. Polynucleic acid (NA) includes DNA, and nucleotides include dNTPs.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Throughout this application, where publications are referenced, the disclosures of these publications in their entireties are hereby incorporated by reference into this application to more fully describe the state of the art to which this invention pertains.

Single base resolution DNA sequencing is a significant goal within biotechnology. To date the majority of techniques require either reconstruction of the sequence from small reads or many repeated runs to achieve fidelity. Sequencing by synthesis (SbS) is the gold standard among currently employed DNA sequencing methods, thanks to its high accuracy. It employs the incorporation of nucleotides modified at the base with a fluorescent label and at 3′-OH position with a protecting group. These modified nucleotides are known as reversible terminators. DNA sequence is determined by synthesizing a complimentary strand of DNA alongside the template strand by adding one modified nucleotide per synthesis cycle. Each cycle consists of incorporation of a modified nucleotide in a growing DNA strand, optical imaging to identify the type of nucleotide, and removal of labels and protecting groups, thus making 3′-OH group available for addition of the next nucleotide.

The main limitation of SbS methods is long cycle time, mainly due to the need to optically image a large area of a flow cell. Accumulation of “scars” (parts of the linkers connecting labels to the bases that remain on the growing DNA strand after removal of the labels) leads to eventual termination of synthesis of the complimentary strand, thus limiting the size of sequenced fragments to ˜150 bases. Other established sequencing methods offer longer read length and faster sequencing times.

Single-molecule real time (SMRT) sequencing technology such as that developed by PacBio uses an enzyme immobilized to the surface of a zero-mode waveguide (ZMW). When a sequencing reaction begins, the enzyme incorporates nucleotides modified with fluorophores attached to the triphosphate group into the growing DNA strand. When the labeled nucleotide momentarily “pauses” in the active pocket of polymerase, it generates a stronger fluorescent signal compared with the background signal from free-floating modified nucleotides in solution. The high-resolution camera of the ZMW records the fluorescence of the successive nucleotides being incorporated in a movie-like fashion. After each nucleotide incorporation the fluorophore is released from the growing DNA chain, together with a pyrophosphate group, through triphosphate hydrolysis, which leaves behind no “scars”, so the next nucleotide with a fluorophore can come in. Thanks to a special library preparation technique, the same circular DNA fragment can be read multiple times, thus overcoming the high error rates associated with real-time sequencing.

Semiconductor sequencing technologies represent affordable and rapid benchtop sequencing systems. Technology such as that developed by Ion Torrent uses an array of semiconductor chips to detect nucleotide incorporation events by sensing small pH changes. This technology requires no specialized enzymes and no modification of native nucleotides.

Nanopore sequencing platforms such as that developed by Oxford Nanopore Technologies records changes in current through a biological nanopore as the result of nucleic acids passing through it. Each of the nucleotides has a unique current modulation signature and can be identified without any labels. This approach offers fast sequencing of long DNA fragments, but hurdles remain in achieving single base resolution and high enough accuracy.

There is thus a need for technologies that combine the scalability and speed of semiconductor-based electrical detection with the high accuracy of SbS technologies to provide long reads at single base pair resolution.

Provided herein, are systems and methods for nucleic acid sequencing that address the needs described above. The systems and methods include an electrochemical nanoelectrode sensor, a biological polymerase to bring a polynucleotide strand into the proximity of the sensor, and modified nucleotides bearing electroactive labels. The labels may be covalently attached to 3′-OH group of the nucleotides via a cleavable ester group. During incorporation of a modified nucleotide into a growing nucleic acid chain, the electroactive label may “idle” inside the sensor generating a strong signal until the esterase function of the polymerase cleaves the ester group, thus removing the label away from the sensor. The benefit of using 3′-esterified nucleotides is that the relatively slow cleavage of 3′-ester group by the polymerase provides the electroactive label with more residence time at the sensor, resulting in a stronger signal. Scarless removal of labels from the growing nucleic acid strand may allow for longer reads.

The systems and methods provided herein may include a device that can read long reads with single base pair resolution. The present disclosure may also incorporate the addition of a translocating protein such as a biological polymerase as a method to bring a polynucleotide strand into the probing device described in U.S. Pat. No. 11,131,646, issued on Sep. 28, 2021, which is incorporated in its entirety by reference. The benefit of this modality is that the translocating protein may act as a controlled localization site to bring the polynucleotide strand into the sensing zone and at the same time provide a controlled rate of translocation within the sensing zone, which are two parameters to be controlled for single base resolution sequencing. The device may be an electrochemical sensor. Hereinafter the terms device or electrochemical sensor may be used interchangeably to refer to the disclosed device.

The electrochemical sensor may comprise a first electrode and a second electrode separated by a nanoscale thick dielectric layer. The first and second electrodes may be held at two different potentials to induce electron transfer in the presence of an electroactive molecule. The dielectric layer provides a small space between the first and second electrodes. This space is small enough for an electroactive molecule to interact with the first and second electrodes directly or indirectly to complete an electrical circuit. The small space between the first and second electrodes over the dielectric layer is hereinafter referred to as a sensing zone. While a redox molecule resides in the sensing zone, electrons flow between reducing and oxidizing electrodes, producing an amplified current signal, which is much higher than a signal expected from a single electron transfer event. This mechanism is different from diffusion-based electron transfer, where electroactive molecules diffuse between the electrodes to produce a measurable electrical signal. The amplified current signal produced by the electron flow generated when a redox molecule resides in the sensing zone may be measured and used to identify the presence of a redox molecule in the sensing zone.

The size of the electrodes should be small to limit the background signal coming from free labeled nucleotides in solution. The preferred electrode size in contact with the solution may be between 50 nm by 10 nm and 5 micron by 1 micron.

The critical parameters are the dielectric thickness and the overlap between the two working electrodes. The dielectric thickness should be small enough to allow electron transfer in the presence of an electroactive label while providing sufficient insulation between the electrodes to avoid shorting. The preferred thickness of the dielectric layer may be from about 1 nm to about 10 nm. For example, the thickness of the dielectric layer may about 0.5 nm, 1 nm, 1.25 nm, 1.5 nm, 1.75 nm, 2 nm, 2.25 nm, 2.5 nm, 2.75 nm, 3 nm, 3.25 nm, 3.5 nm, 3.75 nm, 4 nm, 4.25 nm, 4.5 nm, 4.75 nm, 5 nm, 5.25 nm, 5.5 nm, 5.75 nm, 6 nm, 6.25 nm, 6.5 nm, 6.75 nm, 7 nm, 7.25 nm, 7.5 nm, 7.75 nm, 8 nm, 8.25 nm, 8.5 nm, 8.75 nm, 9 nm, 9.25 nm, 9.5 nm, 9.75 nm, or about 10 nm. The overlap between the electrodes should be kept to a minimum to avoid shorting through pinholes in the dielectric layer.

Non-limiting examples of suitable materials for the electrodes may include platinum, palladium, and titanium nitride.

Non-limiting examples of suitable materials for the dielectric may include hafnium and zirconium silicates, metal oxides or nitrides, such as aluminum oxide, titanium dioxide, hafnium oxide, zirconium oxide, silicon oxide, silicon nitride, and hexagonal boron nitride.

Non-limiting examples of methods that may be used for fabricating the sensing structure are described in U.S. Pat. No. 11,131,646, which is incorporated in its entirety by reference. For example, fabrication of the sensing structure may include sputtering or evaporation of electrode material and the use of a chemical and/or physical dry etch, or a wet etch, to form the desired electrode shape. A thin dielectric layer between the electrode pairs may be deposited using atomic layer deposition between 1 nm and 10 nm thick. An opening may be etched with a physical and chemical dry etch including but not limited to an ion beam etch, inductively coupled plasma reactive ion etch, or a magnetically enhanced reactive ion etch to expose the nanogap. Arrays of these sensors may be fabricated through this method to increase throughput and base calling accuracy.

The systems and methods provided herein may utilize a biological polymerase that replicates DNA by incorporation of new nucleotides as a way of localizing an electroactive label in the sensing zone of an electrochemical sensor. Prior approaches use gamma-phosphate-modified nucleotides. The present systems and methods instead use 3′-O-modified nucleotides and ester functionality which achieves the result of slowing the kinetics of nucleotide incorporation, thus achieving single base resolution.

In an embodiment, an enzyme that can translocate a polynucleotide strand and incorporate free nucleotides into a polynucleotide strand (a DNA polymerase for example) may be immobilized to the surface of a dielectric between two electrodes. Two different potentials may be applied to each of the electrodes. While the labeled nucleotides floating in the solution generate a background signal, this signal may be removed by passivating the surface of the electrodes after enzyme immobilization to reduce the active electrode area outside the spot where the polymerase is attached. Alternatively, the signal from free-floating labeled nucleotides may be removed by electronic filters. Since the free-floating molecules are under diffusion much faster than the typical sampling frequency of the electronic readout, their activity will manifest as a time-averaged effect (for example a constant DC baseline) which may be removed by filtering the slow response. In contrast the nucleotides that are being incorporated may remain in the sensing zone for a longer period of time and therefore produce transient events that may be detected by the electronic readout as incorporation signal. After the ester bond is hydrolyzed through the action of the enzyme, the label may be removed from the sensing zone and the signal may fall back down to background diffusion level, until the next labeled nucleotide enters the active pocket of the enzyme. The sensor may record the signal as a change in current signal. The type of nucleotide being incorporated may be deduced based on the voltages applied to the electrodes and the signal signature generated by the label.

Non-limiting examples of redox molecules that may be used as labels are metal-organic complexes, such as ferrocene and its derivatives, osmium and ruthenium complexes, conjugated organic molecules, such as tetrathiafulvalene, methylene blue, anthraquinone, phenothiazine, aminophenol, nitrophenol, erythrosine B, ATTO MB2, etc. The redox species must undergo reversible oxidation-reduction reactions under applied electrical potential to enable the redox detection principle.

The enzyme utilized must be able to incorporate 3′-O-modified dNTPs and subsequently cleave the ester bond to release a free 3′-OH to enable addition of the next nucleotide. Non-limiting examples of suitable enzymes include A-family polymerases which incorporate (2-aminoethoxy)-3-propionyl-dNTPs and allow for continuous DNA synthesis and certain B-family polymerases. A-family polymerases include BF, Bsu, KF, Taq, Tfl, Tth, T3, T5, T7, and EcPol I, Pol γ, Pol θ, and Pol v. 9°N DNA and Vent polymerase may be suitable B family polymerases.

In various embodiments, enzymes with altered activity including but not limited to elevated esterase speed or base specificity may be generated by directed evolution methods. Nucleobases which code for amino acids that are present in the active center of the enzyme of interest may be randomized, whereas other nucleobases are not randomized. Such partially randomized libraries can be screened in vitro by droplet-based microfluidic screening, or via in vivo hypermutation. In this way, enzymes having an altered activity of interest may be identified.

Nucleotide reversible terminators with different 3′-O-blocking chemical groups are described in the prior art and have been incorporated into commercial products for SbS. Examples of such nucleotide reversible terminators include allyl, azidomethyl, (2-aminoethoxy)-3-propionyl, tert-butylthiomethyl, amino, or 2-nitrobenzyl groups. However, there are no known reports of attaching an electroactive molecule via 3′-ester group of dNTPs and its successful incorporation by a polymerase.

Some systems may attach a label to the 2′-OH group of the sugar ring. Other systems may require an additional modification of dNTPs at 3′-OH with a blocking group to render the dNTPs reversible terminators. This method additionally requires a hydrolysis step to release redox moieties before they can be detected. Lastly, this method requires an additional step to chemically cleave the blocking group and release a free 3′-OH to continue the sequencing.

In preferred embodiments, the electroactive molecule is attached (e.g., via covalent bonding) at 3′-OH group of the sugar ring. Attaching the electroactive molecule to 3′-OH group of the sugar ring eliminates the need to further modify the dNTPs by attaching a blocking group to 3′-OH group of the sugar ring to render the dNTPs reversible terminators. In fact, polymerization does not have to be terminated after each addition of the modified nucleotide in the systems and methods described herein. The continuous synthesis achieved by the systems and methods described herein achieves faster sequencing. Additionally, the systems and methods described herein do not require a hydrolysis step to release redox moieties before they can be detected. Instead, the systems and methods described herein produce a signal from a redox label while the label is still incorporated into the enzyme-template-dNTP complex and is being paused within the sensing zone.

Rather than requiring an additional step to chemically cleave the blocking group and release free 3′-OH to continue the sequencing, the systems and methods described herein combine the release of a redox label and the unblocking of 3′-OH via the action of the enzyme itself without requiring any additional steps or chemical reagents, thus streamlining the process.

Rather than having only one type of dNTP (either dATP, dGTP, dCTP, or dTTP/dUTP) being modified per DNA extension cycle, the systems and methods described herein allow for all four types of dNTPs to be modified in a single sample, thus reducing the number of cycles required to sequence a strand of DNA fourfold.

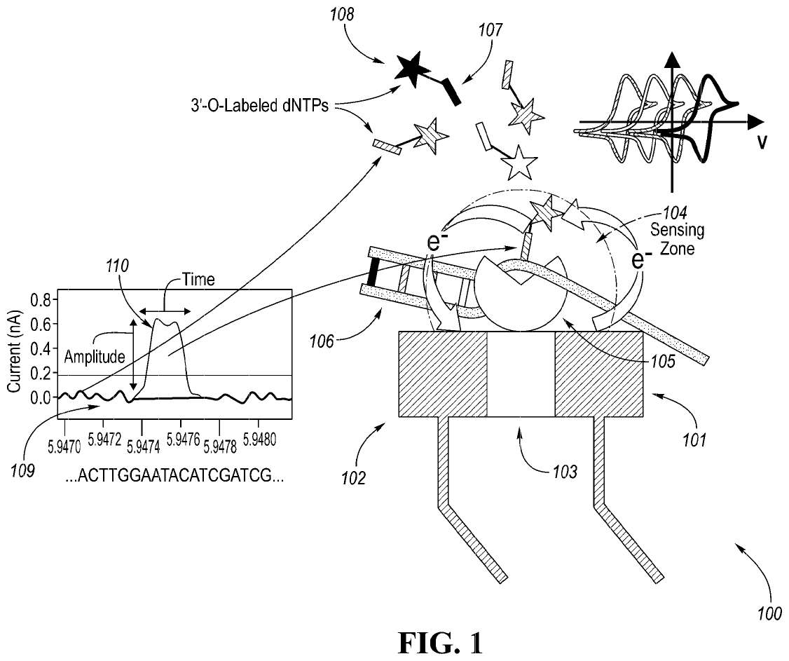

FIG. 1 illustrates an embodiment of a device for sequencing nucleic acids 100 according to the systems and methods described herein. The device comprises a first electrode 101, a second electrode 102, and a dielectric layer 103 positioned between the first and second electrodes. The device may also be referred to as an electrochemical sensor, nanogap sensor, or nanoelectric sensor. The space between the first electrode 101 and the second electrode 102 defines a sensing zone 104. A protein 105 that can translocate a polynucleotide strand and incorporate free nucleotides into the polynucleotide strand is immobilized on the surface of the dielectric layer 103. A solution containing a polynucleotide strand 106 and free-floating modified nucleotides 107, each nucleotide labeled with an electroactive molecule 108, is added to the device 100. The electroactive molecule 108 may be covalently bonded to 3′-OH group of the sugar of the nucleotide. A different potential is applied to each of the first electrode 101 and the second electrode 102. Upon application of the potentials to the first and second electrodes, free-floating modified nucleotides 107 may produce a background signal 109. In contrast, modified nucleotides 107 incorporated into the polynucleotide strand remain in the sensing zone for a longer period of time than the free-floating modified nucleotides 107. The modified nucleotides 107 being incorporated therefore produce transient events during incorporation that can be detected by the electronic readout as an incorporation signal 110. The type of modified nucleotide 107 being incorporated is deduced based on the voltages applied to the electrodes and the signal signature generated by the particular label. The sequence of the polynucleotide strand 106 is thereby deduced from the signal signatures generated by the label on each nucleotide.

According to certain embodiments, the protein immobilized to the surface of the dielectric layer may be an enzyme that can incorporate 3′-O-modified dNTPs and subsequently cleave the ester bond to release a free 3′-OH to enable addition of the next nucleotide. Hereinafter the terms protein, enzyme, or polymerase may be used interchangeably to refer to the protein immobilized to the surface of the dielectric layer 103.

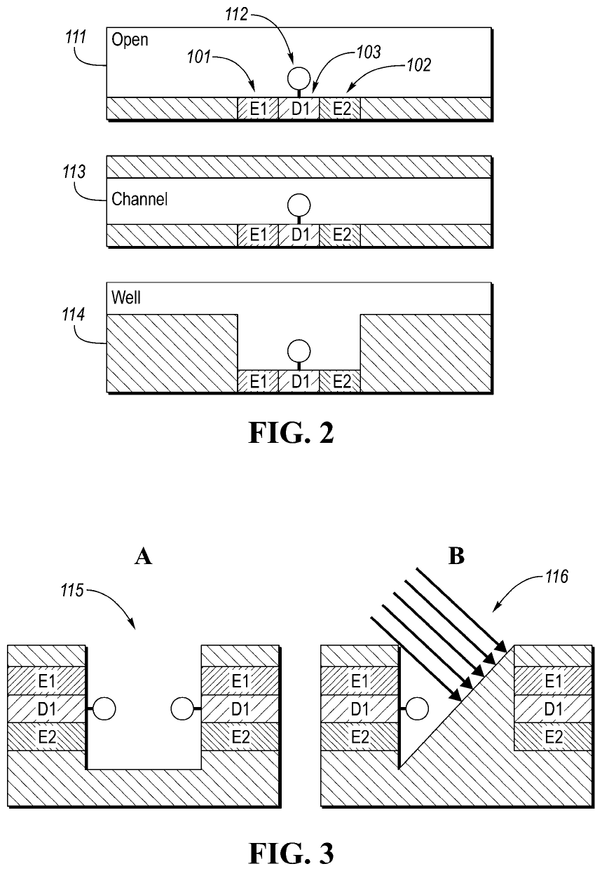

FIG. 2 illustrates various embodiments for the device, wherein the device comprises planar electrodes. The device may have an open configuration 111, wherein the first electrode 101, the dielectric layer 103, and the second electrode 102 are arranged linearly along a surface. Solution containing a polynucleotide strand and modified nucleotides may then be flowed over the surface containing the electrode pair and the dielectric layer with the polymerase 112 attached to the dielectric layer 103. In another embodiment, a channel 113 may be formed, wherein the first electrode 101, the dielectric layer 103, and the second electrode 102 are arranged linearly along a wall of the channel. Solution containing a polynucleotide strand 106 and nucleotides may then be flowed through the channel. In yet another embodiment, a well 114 may be formed in the device. The first electrode 101, dielectric layer 103, and the second electrode 102 may be arranged linearly along the bottom surface of the well as depicted in FIG. 2. Solution containing a polynucleotide strand 106 and nucleotides may then be added to the well. Referring still to FIG. 2, E1 refers to the first electrode 101, E2 refers to the second electrode 102, and D1 refers to the dielectric layer 103.

FIG. 3 illustrates multiple embodiments for the device using a non-planar design for the electrodes. Referring to FIG. 2, E1 refers to the first electrode 101, E2 refers to the second electrode 102, and D1 refers to the dielectric layer 103. In an embodiment, a well 115 may be formed in the device. In this embodiment, the first electrode 101, the dielectric layer 103, and the second electrode 102 are fabricated as a stack in a well format. Two stacks may be fabricated, one on the interior of each wall of the well. In another embodiment, a stack may be fabricated on each wall of the interior of the well 116. Only one stack is accessible by a liquid sample. The accessible stack may have a polymerase attached to the surface of the dielectric layer.

In various embodiments, the modified nucleotide 107 labeled with an electroactive molecule 108 has the following formula:

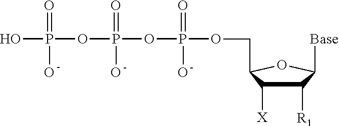

wherein:
   X is

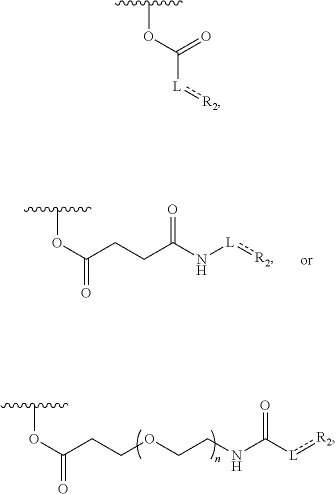

[symbol] is a single bond, a double bond, a triple bond.
Base is Adenine (A), Cytosine (C), Guanine (G), Thymine (T), or Uracil (U),
L (Linker) is absent or a hydrocarbon chain comprising between 1 and 1000 atoms which may contain heteroatoms such as O, N, and S,
n=1-1000
R₁ is H or OH, and
R₂ is a redox label.

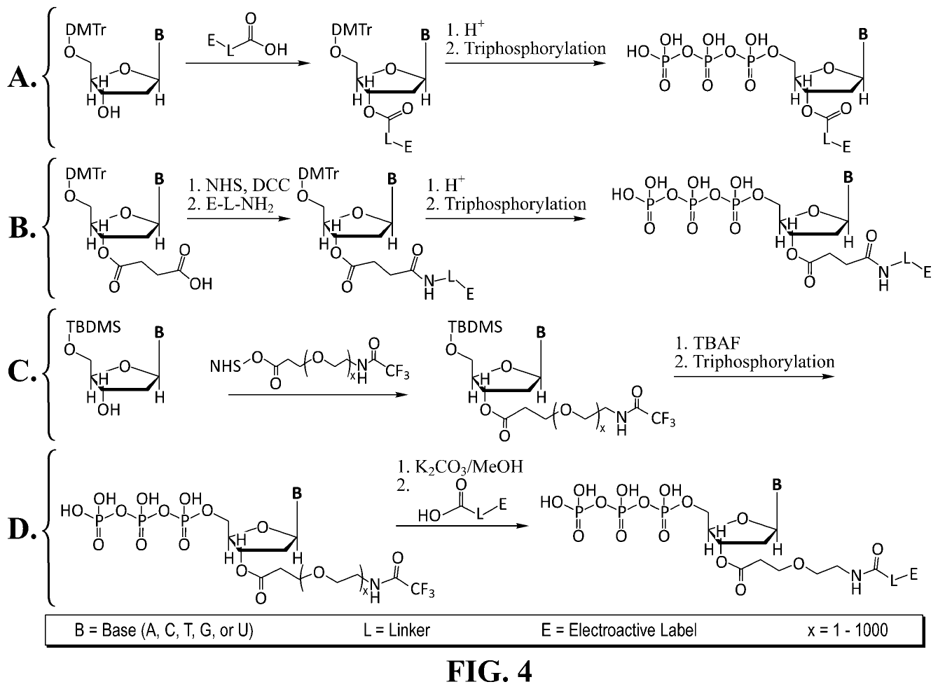

FIG. 4 outlines synthetic strategies to produce 3′-esterified dNTPs with electroactive labels. Route A begins with a 5′-protected nucleoside reacting with a carboxylic acid compound incorporating an electroactive molecule and a linker to form a 3′-esterified nucleoside. This nucleoside is deprotected and converted into a triphosphate in the next step. Alternatively, 5′-O-dimethoxytrityl-dN-3′-O-succinic-acid can be used as the starting material, and an electroactive molecule with an amino group can be coupled to the protected nucleotide derivative, followed by deprotection and triphosphorylation (Route B). With Route C, 3′-ester may be formed first, followed by triphosphorylation, and finally attaching an electroactive molecule via amide formation.

Examples of modified nucleotides 107 labeled with an electroactive molecule 108 include compounds having the following formulas:

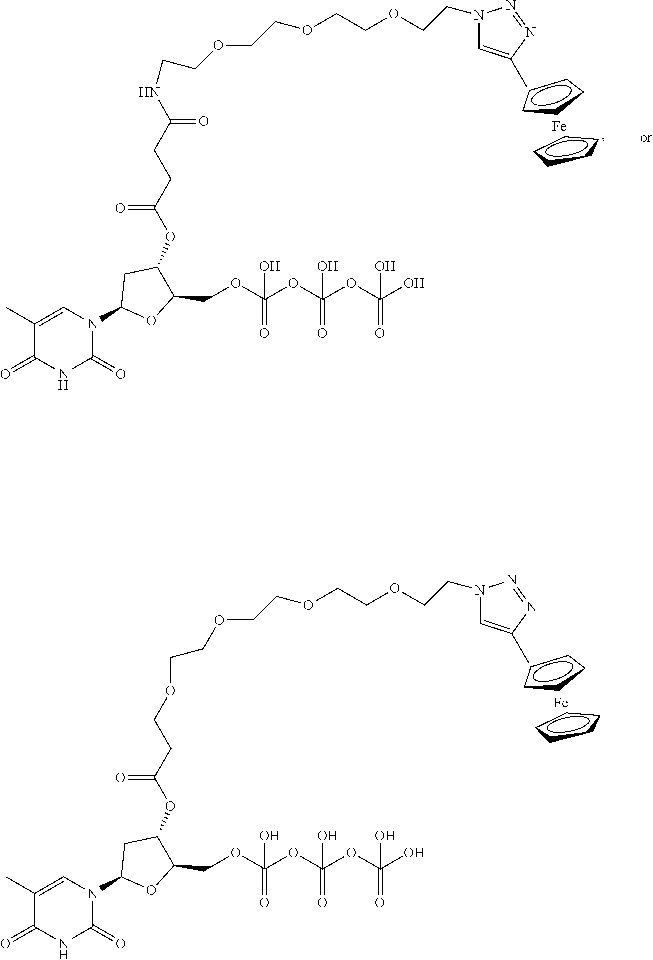

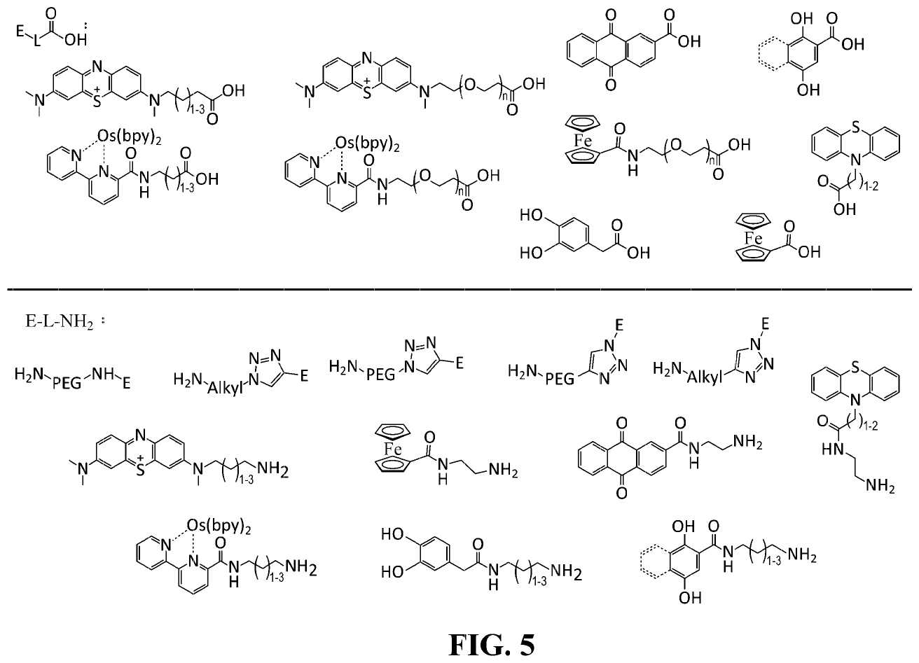

FIG. 5 illustrates examples of electroactive labels and linkers group that may be used to modify the modified nucleotides 107 labeled with an electroactive molecule 108. A linker is a hydrocarbon chain comprising between 1 and 1000 atoms which may contain heteroatoms such as O, N, and S. In the examples in FIG. 5 PEG stands for polyethylene glycol.

In certain embodiments, the systems and methods described herein include nucleotides 107 modified with redox labels 108. The free-floating nucleotides added to a sample may include two, three, or four, different dNTPs (e.g., dATP, dCTP, dGTP, or dTTP/dUTP). Each type of nucleotide may have a distinct redox label. For example, adenine may be modified to include a redox label and cytosine may be modified to include a different redox label. A sample may include a mixture of labeled and unlabeled nucleotides. For example, dATP, dCTP and dGTP may each be labeled with distinct redox labels and dTTP/dUTP may be modified with a 3′-ester moiety but have no electroactive label. Alternatively, a device may include more than one well or channel. In one well, a sample may include labeled dTTP and dCTP with the labels for each nucleotide having distinct electrochemical signatures. In a second well, a sample may include dATP and dGTP having labels distinct from one another. Electrochemical measurements from the samples in well 1 may be overlayed with that of the samples in well 2 to reconstruct the full sequence of a polynucleotide strand during post-processing steps. General examples of how a strand of DNA is replicated to incorporate redox-modified nucleotides are provided in U.S. Pat. No. 11,131,646, which is incorporated herein by reference in its entirety.

The systems and methods described herein include an enzyme attached to the surface of a dielectric layer. The following non-limiting methods for conjugating an enzyme to the surface of the electrochemical sensor include: using bifunctional coupling agents that react with the dielectric material, such as silicon oxide or aluminum oxide, on one end (for example silane chemistry or organophosphorous acids chemistry) and biomolecules on the other end (for example carboxyl, aldehyde, sulfonic acid, isothiocyanate, NHS ester, epoxide, azlactone, or carbodiimide chemistry); using a double-stranded DNA molecule as a coupling agent between the dielectric and the enzyme; physically adsorbing a polymerase onto the dielectric layer, rather than covalently attaching it; or using a streptavidin-biotin pair for non-covalent attachment.

When using a double-stranded DNA molecule as a coupling agent between the dielectric layer and the enzyme, oligonucleotides with a specific sequence are immobilized on the dielectric material of the electrochemical sensor. When enzymes modified with oligonucleotides with complementary sequence are introduced, they are immobilized to the dielectric material via hybridization.

A key factor in choosing a method for immobilizing the protein to the surface of the dielectric layer is to select a chemistry that is selective for the dielectric material (for example aluminum oxide) over the metal first and second electrodes so that the covalent binding happens on the dielectric between the electrodes and not on top of the electrodes themselves.

An electric field generated by the first and second electrodes during the polymerase immobilization process may be used to guide a polymerase to the dielectric layer and to induce a uniform orientation on the surface. For example, voltages may be chosen to attract an electrically charged polymerase with symmetric forces so that the polymerase binds in between the first and second electrodes. Where a polymerase needs to be selectively adsorbed onto the dielectric layer, the voltages on the first and second electrodes may be set to create a surface charge unfavorable to polymerase attachment to the electrodes (adsorption is reduced when the surface charge matches the isoelectric point of polymerase). Alternatively, a lateral electric field may be created to control the orientation of the polymerase molecules, as uniform orientation can result in improved sensor performance.

In one embodiment, a protein is attached to the surface of the electrochemical sensor via any of the techniques described above, then a polynucleotide strand, primers, and redox-modified dNTPs are added to the sensor to form a polynucleotide strand-enzyme-dNTP complex. The sequencing reaction may then begin. In alternative embodiments, a polynucleotide strand, enzyme, and nucleotide reversible terminators (dNTPs modified with removable blocking groups that prevent polynucleotide strand extension) may be brought together off of the sensor to form the polynucleotide strand-enzyme-dNTP complex. The pre-formed complexes are then attached to the sensor surface. To begin sequencing, a blocking group of the nucleotide reversible terminator is released. In one or more embodiments, this blocking group is not an ester groups, but rather may be chosen from either commercially available or novel chemistries. Suitable non-limiting chemistries for the blocking group include3′-O-allyl, 3′-O-azidomethyl, and 3′-ONH₂ groups.

To accurately determine the sequence of a polynucleotide strand using the systems and methods described herein, only one polynucleotide strand may be sequenced per electrochemical sensor. This can be controlled by providing a low concentration of enzyme during the surface attachment step, such that statistically it is only possible to have either 0 or 1 enzyme per sensor. Alternatively, the concentration of target polynucleotide strands may be kept low compared to the number of available enzymes.

In other embodiments, a single polynucleotide strand may be pre-loaded onto a larger entity. For example, a single polynucleotide strand may be pre-loaded onto a bead of adequate size that fits into a well containing a sensor with many polymerases. Only one bead may fit into each well. The bead may be magnetic and thus actuated in a magnetic field. Once the bead has entered the well the polynucleotide strand can be released from the bead by an external stimulus, such as light, temperature, or pH and directly attracted to the sensor surface via an electric field. A linker specifically designed to release the polynucleotide strand from the bead upon application of a particular external stimulus may be used to attach the polynucleotide strand to the bead. The unloaded bead may then be released and the next loaded bead from the prepared library may be moved into the well.

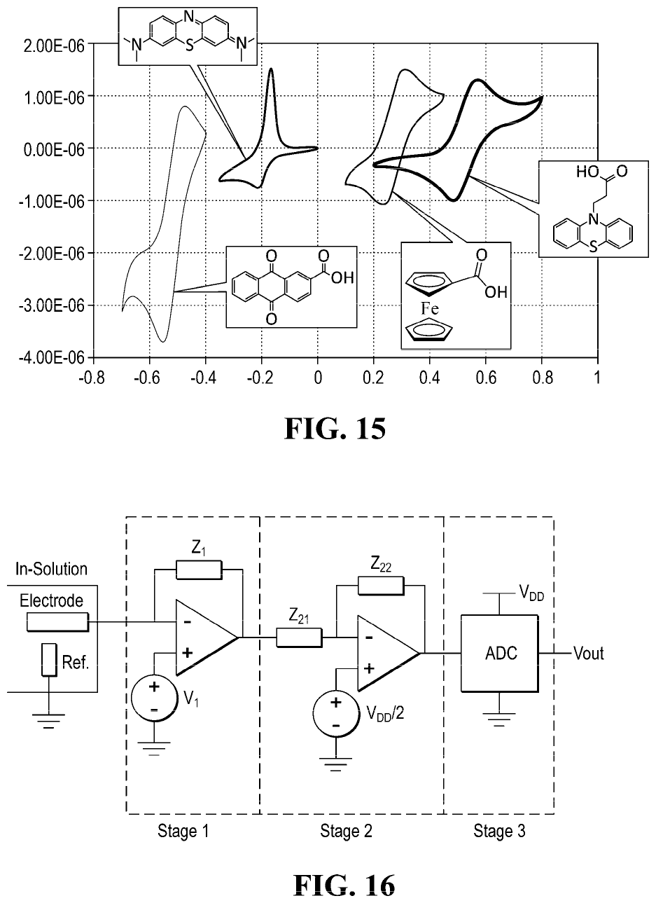

In certain embodiments, when an enzyme-polynucleotide strand-nucleotide complex is formed, an electroactive molecule facilitates electron transfer between the first and second electrodes. The change in the current signal may be used to identify the identity of the nucleotide base being incorporated. To identify each unique base being incorporated, each type of nucleotide is modified with a different electroactive label. All four labels may have unique electrochemical properties and may be distinguishable from each other based on their current-voltage dependence (I-V curve, such as a square wave voltammetry or cyclic voltammetry) or based on the amount of current they produce upon interacting with the electrodes. Examples of distinguishable electroactive labels and their cyclic voltammograms are illustrated in FIG. 15. Thus, it is feasible to have each of the four types of nucleotides modified with a different redox label. To achieve labels producing various amounts of current, different quantities of identical electroactive molecules can be attached through a linker with multiple functional groups, i.e., a dendrimer or a polymer.

In alternative embodiments, three out of four types of nucleotides may be labeled with distinguishable electroactive labels, while the fourth nucleotide is modified with a 3′-ester moiety but no electroactive label. A lack of electrochemical signal in this case may signify the incorporation of the fourth type of nucleotide. At a minimum, two differently labeled types of non-labeled nucleotides may be used for sequence reconstruction.

In certain embodiments, the electrochemical sensor comprises two electrodes that are separated by a dielectric layer. For each electrode, the voltage V₁ is applied to the first electrode and the voltage V₂ is applied to the second electrode. Current then flows from each of the first and second electrodes to an electronic module designed to sense the currents. The currents may be positive or negative. When an electroactive molecule enters the sensing zone between the first and second electrodes, it may temporarily induce higher current signals of the opposite polarity on each electrode. This is the signal of interest.

Differential DC currents (from tunneling or from currents flowing via the polymerase in the bulk solution) may overload the signal path of the frontend. Therefore, the DC gain of readout electronics may be kept low enough so that the output does not rail due to high DC inputs. Additionally, the AC signals of interest may be amplified higher than the parasitic DC signals to improve the readout.

FIG. 16 illustrates an embodiment of readout electronics shown for one electrode. This embodiment has three stages. The third stage comprises an analog-to-digital (AD) converter that converts the analog voltage signal to a digital readout. The precision of the AD converter is set by the ratio of the signal-to-noise at the input. The function of the first two stages is to separate the DC from the AC input signal and to amplify the AC signal. The gain of the second stage can be set such that the noise of the AD converter is negligible in comparison to the amplitude of the AC signal. The first stage sets the voltage of the electrode, which is necessary for oxidation or reduction to occur. The feedback impedance, Z₁, is chosen such that even the largest DC input signal will not overload the output of the first stage. The impedances in the second stage are chosen in a way that only the AC signals are amplified. In alternative embodiments, Z₂₁ may be purely capacitive. In other embodiments, with regard to Z₂₂, a resistor and a capacitor may be in parallel. In another embodiment, the capacitance of Z₂₁ may be larger than Z₂₂ and the ratio of the two capacitors may set the gain of the second stage.

An additional method to increase the signal-to-noise ratio is described herein to solve the problem of higher noise typically exhibited by electronic devices at lower frequencies. At higher frequencies, the noise floor (i.e. spectral density of noise) is constant. In certain embodiments, the method will include operating at a higher frequency, f_{Carrier}, where the noise floor is lower. For example, the method may include modulating V₁ applied at f_{Carrier} and then using a bandpass filter in the signal chain centered around f_{Carrier}. After the bandpass filter, the signal is demodulated back to the base band.

In various embodiments, the device may comprise an array of individual sensors that are connected electrically.

In another aspect, a system for nucleic acid sequencing is provided. The system includes at least one device that includes a first electrode, a second electrode, and a dielectric layer positioned between the first electrode and the second electrode. A polymerase enzyme is attached to the surface of the dielectric layer. The polymerase enzyme incorporates dNTPs modified with an electroactive label covalently bound to 3′-OH group of the sugar ring of the nucleotide via an ester group into a polynucleotide strand at the dielectric layer and subsequently cleaves the ester bond linking the electroactive label to the nucleotide in order to release a free 3′-OH to enable addition of the next nucleotide. The controller directs exposure of the polymerase enzyme to a sample including the polynucleotide strand and dNTPs modified with an electroactive label covalently bound to 3′-OH group of the sugar ring of the nucleotide via an ester group. Once the polymerase enzyme is exposed to a sample including the polynucleotide strand and the modified nucleotides, the controller applies a first voltage, V₁ to the first electrode and a second voltage V₂ to the second electrode. The controller then induces detection of current flowing from each electrode. The currents detected can be positive or negative. When an electroactive molecule enters the sensing zone between the two electrodes, it temporarily induces higher current signals of opposite polarity on both electrodes. The signal from a first electrode may be the inverse of the signal from a second electrode. The controller measures these signals. The controller may calculate the differential value of the two current signals. This differential value may be used as the final signal for analysis in determining which type of nucleotide is present within the sensing zone.

In principle, conventional targeted or universal library preparation methods may be applied where the amplicons carry known flanking sequences representing universal primer annealing sites for the sequencing-by-synthesis reaction. Exemplary methods for the preparation of template polynucleotide strands include the following three library preparation strategies.

Conventional library prep: DNA is blunt end repaired and dA-tailed to be ligated with dT-tailed pseudo-double-stranded, Y-shaped adapters and amplified via PCR to gain asymmetric flanking sites. Amplicon sizes may range from 50-5000 bp. Adapters can contain unique molecular identifiers to overcome PCR-introduced errors and which allow unique and full DNA reconstruction in case a two redox-label readout is preferred during sequencing. Readout strategies using three or four redox labels are compatible with this library preparation strategy.

2D-readout library prep: DNA is blunt end repaired and dA-tailed to be ligated with dT-tailed pseudo-double-stranded, Y-shaped adapters and hairpin adapters. Then a positive selection process is used to obtain asymmetrically ligated products. These products may be used directly for the sequencing reaction. In the alternative, an amplification can be conducted via multiple primer extension or any other isothermal amplification method, including but not limited to Loop-mediated isothermal amplification (LAMP), Recombinase Polymerase Amplification, or rITA. Product or amplicon sizes are determined by the processivity of the polymerases that are used throughout the process. Adapters may contain unique molecular identifiers to overcome amplification-introduced errors. All readout strategies are compatible with this library preparation strategy. The linkage of the sense and antisense strand may increase the accuracy of the sequencing readout.

Rolling circle library prep: DNA is blunt end repaired and dA-tailed to be ligated with dT-tailed hairpin adapters. These products may be used directly for the sequencing reaction. In the alternative, an amplification may be conducted via rolling circle amplification (RCA). Product or amplicon size are not limited. Adapters may contain unique molecular identifiers to overcome amplification-introduced errors. All readout strategies are compatible with this library preparation strategy. The linkage of sense and antisense strand and the multiplicity of this single locus in one molecule may increase the accuracy of the sequencing readout.

Alternatively, a given DNA sample may also be directly sequenced without prior library preparation by using target specific sequencing primers. The primer annealing to the prepared DNA can take place on the chip. For example, the prepared DNA may be denatured for 1 min at 95° C. and then the primer may be allowed to anneal to the DNA for 1 min at 60° C., provided that the polymerase is capable of withstanding denaturing conditions. In preferred embodiments, the primer is annealed to the prepared DNA strand prior to loading onto the chip, and the primer-template DNA conjugate remains stable until it reaches the polymerase.

Non-limiting polynucleotide strand types that may be sequenced by the systems and methods described herein include DNA and RNA. If RNA is used, the RNA may be processed upstream with a Reverse Transcriptase (RT) enzyme to generate cDNA that may be subsequently read using an immobilized DNA polymerase. Alternatively, the RT enzyme may be immobilized to the surface of the dielectric layer. As the RNA sequence is replicated, the incorporation of redox modified dNTPs by the RT enzyme may be used to determine the original RNA template. To sequence a random mixture of RNA molecules, the RNA may be ligated to a universal sequence which may be used as a docking site for a universal primer at which the sequencing procedure would start.

### EXAMPLES

### Example 1: Synthesis of a Ferrocene-Labeled dUTP Reversible Terminator with Ester group (A3)

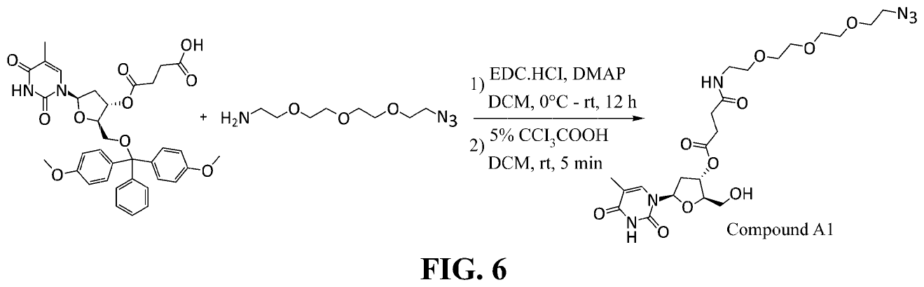

Example 1 shows a method for synthesizing a ferrocene-labeled dUTP reversible terminator with an ester group. The compounds utilized and formed in this method are illustrated in FIGS. 6-8.

Dissolve 5′-O-(4,4′-Dimethoxytrityl)-thymidine-3-O-succinic acid of the acid (500 mg, 0.78 mmol), 1-(3-dimethylaminopropyl)-3-ethylcarbodiimide hydrochloride (EDC·HCL) (208 mg, 1.1 mmol) and 4-dimethylaminopyridine (DMAP) (9.5 mg, 0.078 mmol) in 5 mL of DCM in a 50 mL round bottom flask. Stir continuously for 10 minutes. Dissolve Azide-PEG4-Amine (244 mg. 1.17 mmol) in 1 mL DCM and add dropwise to the stirring solution under ice cold conditions. Bring the reaction mixture to room temperature and keep stirring overnight. Quench the reaction mixture with 25 mL of 1.0 M HCL once the starting material is consumed as indicated by TLC. Transfer the whole solution into a separatory funnel containing 25 mL of DCM and extract the organic layer. Wash the organic layer with another 25 mL 1.0 M HCL. Collect the organic layer and dry it using anhydrous MgSO4. Filter the solution and remove the solvent by vacuum. Dissolve the crude reaction mixture again in 5 mL dry DCM. Slowly introduce 5% trichloro acetic acid into the reaction mixture until the solution becomes bright orange in color. This is an indication of the deprotection of dimethoxytrityl group which may be confirmed by TLC. Evaporate and purify the mixture obtained using flash chromatography on a silica gel (MeOH/CHCl3, 1:10) to obtain compound A1 as a colorless viscous liquid (190 mg, yield 45%) (See for example FIG. 6).

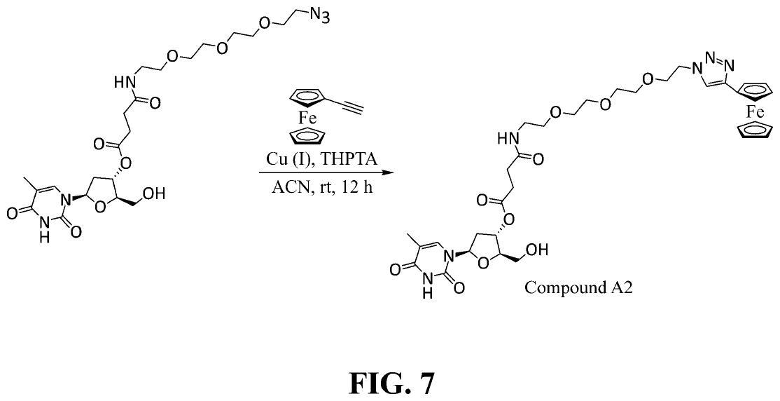

Slowly add a mixture of CuBr (38.7 mg. 0.270 mmol) and tris-hydroxypropyltriazolylmethylamine (THPTA) (118 mg. 0.272 mmol) in 2 mL acetonitrile (ACN) to a solution of compound 2 (50 mg, 0.092 mmol) and ethynyl ferrocene (23 mg. 0.109 mmol) in 1 mL acetonitrile (ACN) under room temperature. Keep stirring the reaction mixture overnight until all of compound 2 is consumed as indicated by TLC. Evaporate the organic solvent and wash the crude mixture with 25 mL of aqueous solution. Extract using 2×25 mL of ethyl acetate. Combine and dry the organic layer using anhydrous MgSO4. Remove the solvent under vacuum and purify the crude mix using flash chromatography on a silica gel (Methanol-Chloroform, 0.5:10) to yield compound A2 as a yellow viscous liquid (27 mg, yield 46%) See for example FIG. 7).

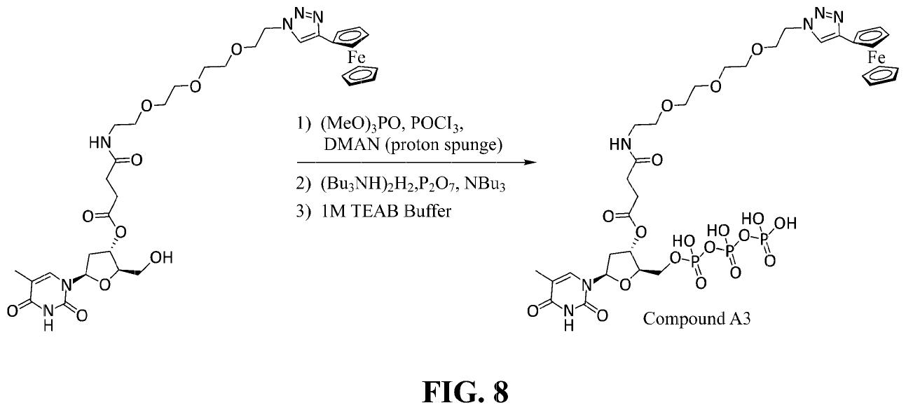

Measure compound 3 (25 mg, 0.033 mmol) and 1,8-bis(dimethylamino) naphthalene (DMAN) (7.2 mg, 0.034 mmol) in a 50 mL round bottom flask and take out tributylammonium pyrophosphate (TBAP) (17 mg, 0.031 mmol) in a separate vial. Keep both for drying under high vacuum over P₂O₅ (ca 500 mg) for next 1 h. Chill Tributyl amine (NBu3) (0.05 mL, 0.210 mmol) to −20° C. After the drying process, perform a vacuum-nitrogen cycle 3 times for both the flask and the vial. Add 0.5 mL of acetonitrile into the vial containing TBAP and chill to −20° C. Then, slowly add 0.5 mL of trimethyl phosphate to the flask containing compound 3 and DMAN, and stir the flask continuously for 10 minutes under a mix of ice and dry ice bath. Then add phosphoryl chloride dropwise to the flask over a period of 5 minutes and keep stirring for another 30 minutes under ice/dry ice bath. Then add chilled NBu3 and TBAP into the reaction mixture and stir for the next 1 h under ice cold conditions. Introduce 1.0 M chilled TEAB Buffer to the reaction mixture and stir for another 1 h. Reduce the solution under vacuum to remove any remaining organic solvent. Freeze the obtained aqueous solution of the reaction mixture at −80° C. for lyophilization. The frozen mixture was lyophilized and purified using reverse-phase HPLC (C-18 column, TEAB/Acetonitrile linear gradient) to afford compound A3 (See for example, FIG. 8).

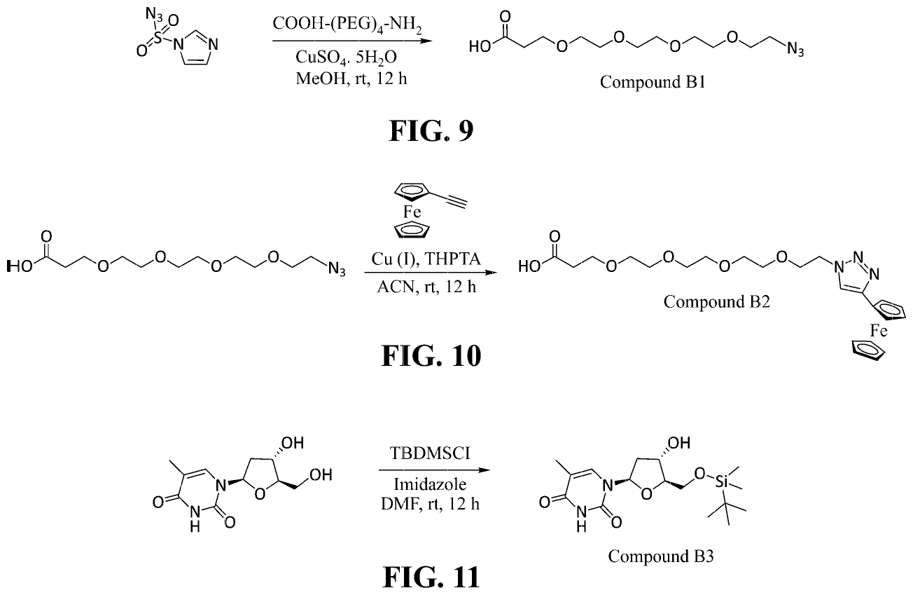

### Example 2: Synthesis of Ferrocene-Labeled dUTP Reversible Terminator with Ester Group (B5). The Compounds Utilized and Formed in this Method are Illustrated in FIGS. 9-13

Sodium Azide (1.3 g. 20 mmol) was taken in a 2-neck 100 mL round bottom flask and oxygen was removed from the reaction environment with 3 cycles of vacuum/argon purging. 20 mL acetonitrile was added to the flask and the mixture was cooled in ice bath for 10 minutes. Sulfuryl chloride (1.6 mL, 20 mmol) was then injected into the flask over a period of 2 minutes. The solution was brought back to room temperature. After 12 h, the reaction flask was cooled on ice bath for 10 minutes. Imidazole (2.6 g, 40 mmol) was then added portion wise to the reaction mixture. The flask was warmed to room temperature and left to stir for next 6 h. 50 mL ethyl acetate was added into the reaction mixture and the entire solution was then transferred to a separatory funnel. The solution was washed with 50 mL water followed by 50 mL sat. NaHCO₃ solution and finally washed with 50 mL brine solution. The organic layer was collected and chilled in an ice bath. HCl in ethanol was prepared by dissolving 2.1 mL CH3COCl in 7.5 mL of ice-cold anhydrous ethanol. The HCl solution was chilled for another 10 minutes under ice bath and then added dropwise to the organic layer upon stirring. The product, imidazole sulfonyl azide, appeared as white precipitate and the suspension was cooled again and vacuum filtered using Hirsch funnel to obtain compound B1 (2 g, yield 58%) (See for example FIG. 9).

Imidazole sulfonyl azide (38 mg. 0.22 mmol) and amine-(PEG) 4-acid (50 mg. 0.19 mmol) were dissolved in 1 mL of methanol in a 25 mL round bottom flask. K2CO3 was then added to the reaction mixture followed by the addition of CuSO4·5H2O in 0.2 mL methanol. The solution turned blue at first and slowly turned to yellow over a period of 1 h. The solution was then kept stirring overnight at room temperature. 5 mL methanol was added to the reaction mixture and filtered the solution using gravity filtration. The solution was then evaporated to remove the solvent. The obtained crude mixture was directly used for the next step without further purification.

A mixture of CuBr (33 mg, 0.23 mmol) and tris-hydroxypropyltriazolylmethylamine (THPTA) (100 mg. 0.23 mmol) in 2 mL acetonitrile (ACN) was slowly added to a solution of crude mix (ca 55 mg. 0.19 mmol) from previous step and ethynyl ferrocene (50 mg. 0.24 mmol) in 1 mL acetonitrile (ACN) under room temperature. The reaction mixture was then kept for stirring overnight until all the compound 2 was consumed as indicated by TLC. The organic solvent was then evaporated, and the crude mixture was washed with 25 mL of aqueous solution and extracted using 2×25 mL of ethyl acetate. The organic layer was combined and dried using anhydrous MgSO4. The solvent was then removed under vacuum and the crude mix was purified using flash chromatography on reverse-phase C-18 column (Water/Acetonitrile, 1:5) to yield compound B2 as a yellow viscous liquid (23 mg, yield 24%) (See for example FIG. 10).

A 50 mL 2-neck round bottom flask was charged with thymidine (100 mg. 0.41 mmol) and imidazole (60 mg. 0.88 mmol) and kept under vacuum for 10 minutes. 1 mL dry DMF was then added to the flask under argon condition and kept stirring for 10 minutes. Tert-butyldimethylsilyl chloride (TBDMSCl) (65 mg. 0.43 mmol) was added portion wise through one neck of the flask while the other neck was kept under a continuous flow of argon. The reaction was kept stirring overnight under room temperature. The reaction was quenched by the addition of 25 mL of DI water once thymidine is fully consumed as indicated from TLC. White precipitate of the product can be observed while quenching. Then the solution was washed with 2×25 mL of ethyl acetate. The ethyl acetate solution obtained will be combined and dried over anhydrous MgSO4. The solution was filtered, and ethyl acetate was removed under vacuum. The obtained crude mix was purified using flash chromatography on basic alumina gel (Methanol/Chloroform 1:10) to obtain compound B3 as a white powder. (112 mg, yield 72%) (See for example FIG. 11).

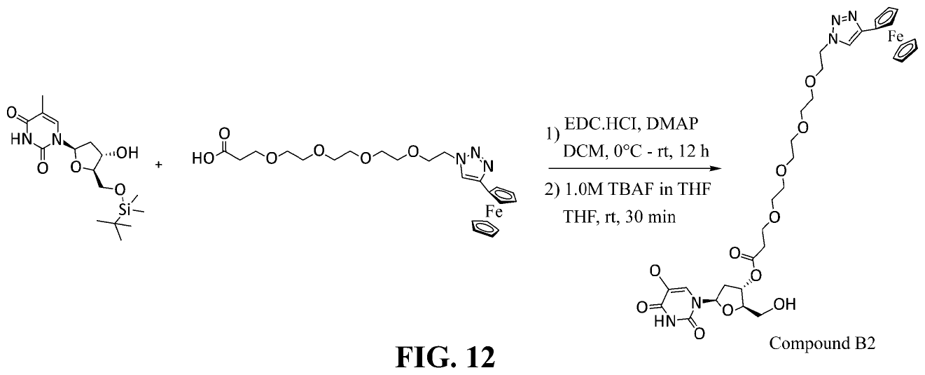

Dissolve 5′-O-tert-butyldimethylsilyl thymidine (20 mg, 0.054 mmol), 1-(3-dimethylaminopropyl)-3-ethylcarbodiimide hydrochloride (EDC·HCL) (14 mg, 0.073 mmol) and 4-dimethylaminopyridine (DMAP) (1 mg, 0.008 mmol) in 5 mL of DCM in a 50 mL round bottom flask. The solution was kept stirring for 10 minutes. Dissolve compound 3 (23 mg. 0.046 mmol) in 1 mL DCM and add dropwise to the stirring solution under ice cold condition. The reaction mixture was brought to room temperature and kept for stirring overnight. Quench the reaction mixture with 25 mL of 1.0 M HCL once the starting material is consumed as indicated by TLC. Transfer the whole solution into a separatory funnel containing 25 mL of DCM and extract the organic layer. Wash the organic layer with another 25 mL 1.0 M HCL. Collect the organic layer and dry it using anhydrous MgSO4. Filter the solution and remove the solvent by vacuum. The obtained crude mixture was purified using flash chromatography on silica gel (MeOH/CHCl3, 1:10) to obtain the intermediate compound (5′-O-tert-Butyldimethylsilyl thymidine-PEG4-Ferrocene). The obtained product was redissolved in 1 mL anhydrous THF. 1.0 M tetrabutyl ammonium fluoride (TBAF) (0.2 mL) in THF was added dropwise to the above solution and kept stirring for 30 minutes. After the reaction was completed as indicated from TLC, the solvent was evaporated, followed by addition of 20 mL saturated NaHCO₃ solution. The solution was extracted with 2×20 mL ethyl acetate. The organic layer was combined and dried using anhydrous MgSO4. The solution was filtered, and the solvent was removed to obtain compound B4 as a yellow viscous liquid (7 mg, yield 21%) (See for example FIG. 12).

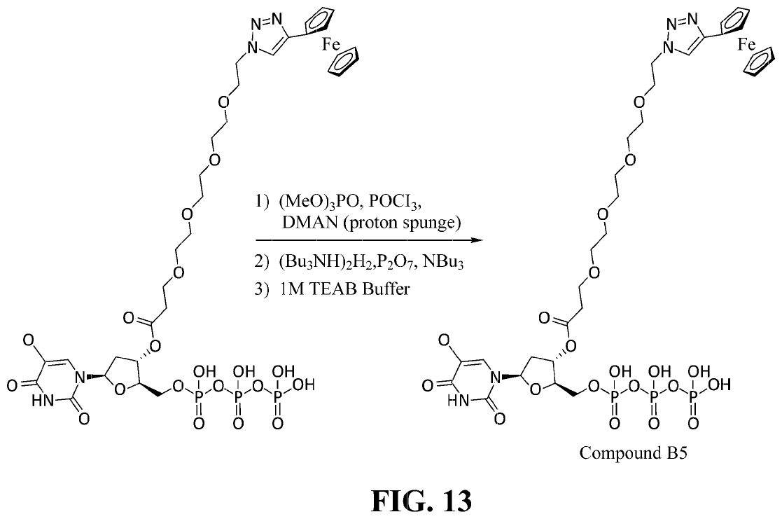

Compound B5 was obtained through triphosphorylation of B4 by following the same experimental procedure as for compound A3 (See for example FIG. 13).

### Example 3: Synthesis of Ferrocene-Labeled dTTP Reversible Terminator with Ester Group and No Linker (C3)

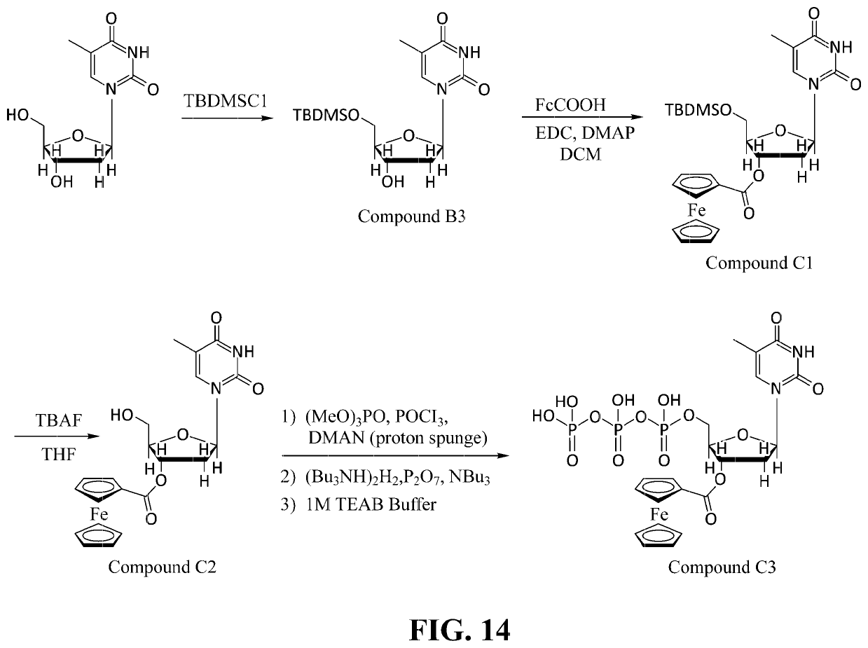

As illustrated in FIG. 14, Example 3 shows a method for synthesizing a ferrocene-labeled dTTP reversible terminator with an ester group and no linker (C3).

Example compound C3 was synthesized using procedures similar to the procedures described in Examples 1 and 2. Compound B3 (0.31 g, 0.87 mmol), Ferrocene carboxylic acid (0.1 g. 0.435 mmol), EDC (0.092 g. 0.048 mmol), and DMAP (0.0026 g. 0.021 mmol) were dissolved in 12 mL of dry DCM in oxygen-free atmosphere and stirred at room temperature. Conversion of compound B3 was monitored by TLC using Hexane/Ethyl Acetate as eluent. After the reaction was completed, the solvents were evaporated and the product was purified using silica gel flash chromatography with Hexane/Ethyl Acetate linear gradient. The yield of the isolated product: 0.06 g. Compound C1 was deprotected to obtain compound C2 by following the same procedure as for compound B4. The final compound C3 was obtained after a triphosphorylation procedure similar to that used to synthesize compounds A3 and B5.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the present disclosure that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A method for nucleic acid sequencing, comprising:
providing at least one device comprising:
a first electrode, a second electrode, and a dielectric layer defining a sensing zone between the first electrode and the second electrode; and
a polymerase enzyme attached to the surface of the dielectric layer, wherein the polymerase enzyme targets a polynucleotide strand to the dielectric layer;
providing to the at least one device a sample including a polynucleotide strand and at least one modified nucleotide, the modified nucleotide having an electroactive label covalently bound to 3′-OH of a sugar ring of the nucleotide via an ester group;
applying a first potential to the first electrode and a second potential to the second electrode to induce electron flow between the first and second electrodes to produce a measurable electrical signal when an electroactive label is present in the sensing zone; and
detecting a first signal from the first electrode and a second signal from the second electrode to determine when a modified nucleotide is present in the sensing zone.

2. The method of claim 1, wherein the polymerase enzyme incorporates a modified nucleotide having an electroactive label covalently bound to 3′-OH of a sugar ring of the nucleotide via an ester group into a polynucleotide strand.

3. The method of claim 2, wherein the polymerase enzyme cleaves the ester group of the modified nucleotide after the nucleotide is incorporated to remove the electroactive label from the sensing zone.

4. The method of claim 3, wherein the polymerase enzyme is one of an A family polymerase or a B family polymerase.

5. The method of claim 1, wherein two or more of dATP, dCTP, dGTP, or dTTP/dUTP is modified with a measurably distinct electroactive label.

6. The method of claim 1, wherein the modified nucleotide having an electroactive label covalently bound to 3′-OH of the sugar ring of the nucleotide via an ester group has the following formula: 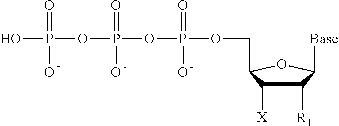
wherein:
X is 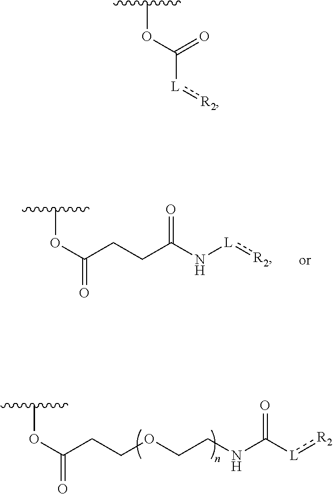 [symbol]
is a single bond, a double bond, a triple bond,
Base is Adenine (A), Cytosine (C), Guanine (G), Thymine (T), or Uracil (U),
L (Linker) is absent or is a hydrocarbon chain comprising between 1 and 1000 atoms which may contain heteroatoms such as O, N, and S,
n=1-1000
R₁ is H or OH, and
R₂ is a redox label.

7. The method of claim 1, further comprising calculating a differential value between the first signal and the second signal to determine when a modified nucleotide is present in the sensing zone.

8. A system for nucleic acid sequencing comprising:
at least one device including:
at least one electrochemical sensor comprising a first electrode, a second electrode, and a dielectric layer defining a sensing zone between the first electrode and the second electrode; and a polymerase enzyme attached to the surface of the dielectric layer, wherein the polymerase enzyme targets a polynucleotide strand to the sensing zone; and
a controller configured to:
direct a first current through the first electrode and a second current through the second electrode to induce electron flow between the first and second electrodes to produce a measurable electrical signal when an electroactive label is present in the sensing zone;
provide to the at least one device a sample including a polynucleotide strand and at least one modified nucleotide, the modified nucleotide having an electroactive label covalently bound to 3′-OH of a sugar ring of the nucleotide via an ester group; and
detect the first current flowing from the first electrode and the second current flowing from the second electrode.

9. The system of claim 8, wherein the polymerase enzyme incorporates a modified nucleotide having an electroactive label covalently bound to 3′-OH of a sugar ring of the nucleotide via an ester group into a polynucleotide strand, and wherein the polymerase enzyme cleaves the ester group of the modified nucleotide after the nucleotide is incorporated to remove the electroactive label from the sensing zone.

10. The system of claim 9, wherein the polymerase enzyme is one of an A family polymerase or a B family polymerase.

11. The system of claim 8, wherein the measurable electrical signal is produced while the electroactive label is still incorporated into the enzyme-template-dNTP complex and while the electroactive label is within the sensing zone.

12. The system of claim 8, wherein the controller is further configured to calculate a differential value between the first current and the second current to determine when a modified nucleotide is present in the sensing zone.

13. The system of claim 8, wherein the modified nucleotide having an electroactive label covalently bound to 3′-OH of the sugar ring of the nucleotide via an ester group has the following formula: 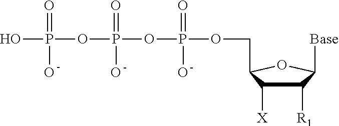
wherein:
X is 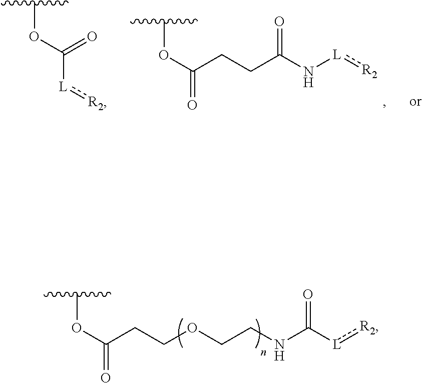 [symbol]
is a single bond, a double bond, a triple bond,
Base is Adenine (A), Cytosine (C), Guanine (G), Thymine (T), or Uracil (U),
L (Linker) is absent or a hydrocarbon chain comprising between 1 and 1000 atoms which may contain heteroatoms such as O, N, and S,
n=1-1000
R₁ is H or OH, and
R₂ is a redox label.

14. The system of claim 8, wherein the at least one device is a plurality of devices.

15. A method for forming a system for nucleic acid sequencing, the method comprising:
providing a first electrode and a second electrode;
positioning a dielectric layer between the first and second electrodes to define a sensing zone;
attaching a polymerase enzyme to the surface of the dielectric layer, wherein the polymerase enzyme incorporates a modified nucleotide having an electroactive label covalently bound to 3′-OH of a sugar ring of the nucleotide via an ester group into a polynucleotide strand; and
configuring the electrodes to detect a change in current when an electroactive label is present within the sensing zone.

16. The method of claim 15, further comprising generating two of dATP, dCTP, dGTP, and dTTP/dUTP each of the two modified with a measurably distinct electroactive label covalently bound to 3′-OH of a sugar ring of the dATP, dCTP, dGTP, and dTTP/dUTP via an ester group.

17. The method of claim 15, further comprising generating dATP, dCTP, dGTP, and dTTP/dUTP each modified with a measurably distinct electroactive label covalently bound to the 3′-OH of a sugar ring of the dATP, dCTP, dGTP, and dTTP/dUTP via an ester group.

18. The method of claim 15, wherein the polymerase enzyme cleaves the ester group of the modified nucleotide after the nucleotide is incorporated to remove the electroactive label from the sensing zone.

19. The method of claim 15, further comprising configuring a controller to:
direct a first current through the first electrode and a second current through the second electrode to induce electron flow between the first and second electrodes;
direct exposure of the polymerase enzyme to a sample including the polynucleotide strand and dNTPs modified with an electroactive label covalently bound to 3′-OH group of the sugar ring of the nucleotide via an ester group; and
detect the first current flowing from the first electrode and the second current flowing from the second electrode.

20. The method of claim 19, wherein the controller is further configured to calculate a differential value between the first current and the second current to determine when a modified nucleotide is present in the sensing zone.
